(19) United States
(12) Patent Application Publication
(10) Pub. No.: US 2023/0013981 A1
(43) Pub. Date: Jan. 19, 2023

(54) WATER-DISPERSIBLE DRY POWDER FORMULATION AND PRODUCTION METHOD THEREOF

(71) Applicant: Genovior Biotech Corporation, Miaoli County (TW)

(72) Inventors: Fu-Ya Wang, Tainan City (TW); Chia-Ann Yang, Miaoli County (TW); Yu-Min Lin, Changhua County (TW)

(21) Appl. No.: 17/372,741

(22) Filed: Jul. 12, 2021

(51) Int. Cl. A61K 9/16; A61K 38/26

(52) U.S. Cl. CPC A61K 9/1623; A61K 38/26; A61K 9/1682; A61K 9/19

(57) ABSTRACT

Disclosed herein is a water-dispersible dry powder formulation that includes, based on the total weight of the water-dispersible dry powder formulation, 0.2 wt % to 4.0 wt % of glucagon, 10 wt % to 95.0 wt % of lactose, 0.001 wt % to 5.0 wt % of acetone, and 0.1 wt % to 10.0 wt % of water. The water-dispersible dry powder formulation has a pH value ranging from 2.0 to 6.0. A method for producing the water-dispersible dry powder formulation is also disclosed.

### FIELD

The disclosure relates to a water-dispersible dry powder formulation and a method for manufacturing the same.

### BACKGROUND

Diabetes mellitus, commonly known as diabetes, is a chronic, lifelong disease associated with high levels of glucose in the blood. Diabetes mellitus may be classified as type I diabetes (or insulin dependent diabetes mellitus, IDDM) and type 2 diabetes (or non insulin dependent diabetes mellitus, NIDDM). Many patients with diabetes self administer insulin to control their blood glucose levels. However, insulin administration might cause a rapid drop in blood glucose (known as hypoglycemia). In patients with type I diabetes, hypoglycemia fails to elicit a normal glucagon response, increasing the risk of severe hypoglycemia. Severe hypoglycemia might cause serious issues such as seizures, unconsciousness, brain damage, or even death.

Administration of glucagon is an established therapy for treating severe hypoglycemia. Emergency glucagon administration can restore normal glucose levels within minutes of administration. However, pharmaceutical preparations containing glucagon have several problems. Glucagon possesses poor solubility in aqueous buffers at or near physiological pH values. Furthermore, when formulated at low or high pH, glucagon demonstrates poor chemical stability and poor physical stability (such as aggregation, fibril formation, and gel formation).

### SUMMARY

Therefore, a first object of the present disclosure is to provide a water-dispersible dry powder formulation that can alleviate at least one of the drawbacks of the prior art.

The water-dispersible dry powder formulation includes, based on the total weight of the water-dispersible dry powder formulation:
0.2 wt % to 4.0 wt % of glucagon;
10 wt % to 95.0 wt % of lactose;
0.001 wt % to 5.0 wt % of acetone; and
0.1 wt % to 10.0 wt % of water;

wherein the water-dispersible dry powder formulation has a pH value ranging from 2.0 to 6.0.

A second object of the present disclosure is to provide a method for producing a water-dispersible dry powder formulation, which can alleviate at least one of the drawbacks of the prior art, and which includes:
a) admixing lactose with water at a temperature ranging from 30° C. to 40° C. to obtain an aqueous lactose solution;
b) admixing the aqueous lactose solution with acetone at room temperature, followed by adjusting a pH value of a first mixture thus obtained to pH 2.0 to 4.0 with an acidic aqueous solution, so that an acidic first mixture is formed;
c) admixing glucagon with water at room temperature, followed by adjusting a pH value of an aqueous glucagon solution thus obtained to pH 2.0 to 4.0 with another acidic aqueous solution, so that an acidic aqueous glucagon solution is formed; and
d) admixing the acidic first mixture with the acidic aqueous glucagon solution to obtain a second mixture, followed by subjecting the second mixture to a drying treatment to obtain the water-dispersible dry powder formulation.

### DETAILED DESCRIPTION

It is to be understood that, if any prior art publication is referred to herein, such reference does not constitute an admission that the publication forms a part of the common general knowledge in the art, in Taiwan or any other country.

For the purpose of this specification, it will be clearly understood that the word “comprising” means “including but not limited to”, and that the word “comprises” has a corresponding meaning.

Unless defined otherwise, all technical and scientific terms used herein have the meaning commonly understood by a person skilled in the art to which the present disclosure belongs. One skilled in the art will recognize many methods and materials similar or equivalent to those described herein, which could be used in the practice of the present disclosure. Indeed, the present disclosure is in no way limited to the methods and materials described.

For the purposes of this specification and appended claims, unless otherwise indicated, all numbers expressing amounts, sizes, dimensions, proportions, shapes, formulations, parameters, percentages, quantities, characteristics, and other numerical values used in the specification and claims are to be understood as being modified in all instances by the term “about” even though the term “about” may not expressly appear with the value, amount or range. Accordingly, unless indicated to the contrary, the numerical parameters set forth in the following specification and attached claims are not and need not be exact, but may be approximate and/or larger or smaller as desired, reflecting tolerances, conversion factors, rounding off, measurement error and the like, and other factors known to those of skill in the art depending on the desired properties sought to be obtained by the presently disclosed subject matter. For example, the term “about,” when referring to a value, can be meant to encompass variations of, in some aspects ±100%, in some aspects ±50%, in some aspects ±20%, in some aspects ±10%, in some aspects ±5%, in some aspects ±1%, in some aspects ±0.5%, and in some aspects ±0.1% from the specified amount, as such variations are appropriate to perform the disclosed methods or employ the disclosed compositions.

The present disclosure provides a water-dispersible dry powder formulation including, based on the total weight of the water-dispersible dry powder formulation:
0.2 wt % to 4.0 wt % of glucagon;
10 wt % to 95.0 wt % of lactose;
0.001 wt % to 5.0 wt % of acetone; and
0.1 wt % to 10.0 wt % of water;

wherein the water-dispersible dry powder formulation has a pH value ranging from 2.0 to 6.0.

According to the present disclosure, glucagon may be present in an amount ranging from 0.5 wt % to 3.5 wt %, based on the total weight of the water-dispersible dry powder formulation.

According to the present disclosure, lactose may be present in an amount ranging from 20 wt % to 90 wt %, based on the total weight of the water-dispersible dry powder formulation.

According to the present disclosure, acetone may be present in an amount ranging from 0.5 wt % to 3.5 wt %, based on the total weight of the water-dispersible dry powder formulation.

According to the present disclosure, water may be present in an amount ranging from 0.5 wt % to 5.0 wt %, based on the total weight of the water-dispersible dry powder formulation.

According to the present disclosure, the water-dispersible dry powder formulation may have a pH value ranging from 2.0 to 4.0.

According to the present disclosure, the water-dispersible dry powder formulation is prepared by a process including the steps of:
a) admixing lactose with water at a temperature ranging from 30° C. to 40° C. to obtain an aqueous lactose solution;
b) admixing the aqueous lactose solution with acetone at room temperature, followed by adjusting a pH value of a first mixture thus obtained to pH 2.0 to 4.0 with an acidic aqueous solution, so that an acidic first mixture is formed;
c) admixing glucagon with water at room temperature, followed by adjusting a pH value of an aqueous glucagon solution thus obtained to pH 2.0 to 4.0 with another acidic aqueous solution, so that an acidic aqueous glucagon solution is formed; and
d) admixing the acidic first mixture with the acidic aqueous glucagon solution to obtain a second mixture, followed by subjecting the second mixture to a drying treatment to obtain the water-dispersible dry powder formulation.

According to the present disclosure, each of the acidic aqueous solutions used in steps (b) and (c) may be an aqueous solution containing hydrochloric acid, formic acid, or acetic acid. The acidic aqueous solutions used in steps (b) and (c) may be the same or different.

According to the present disclosure, in step (d) of the process, the drying treatment is freeze-drying.

The disclosure will be further described by way of the following examples. However, it should be understood that the following examples are solely intended for the purpose of illustration and should not be construed as limiting the disclosure in practice.

### EXAMPLES

### Example 1

### Preparation of Water-Dispersible Freeze-Dried Powder of Present Disclosure

39.2 g of lactose monohydrate was dissolved in 400 g of water for injection (WFI) at a temperature of 30° C. to 40° C., so as to obtain an aqueous lactose solution. 200 g of acetone was added into the aqueous lactose solution, followed by mixing evenly at room temperature. The resultant first mixture was adjusted to pH 2.7 through addition of a 0.05 N HCl solution, so as to obtain an acidic first mixture.

1.07 g of glucagon (Genovior Biotech Co., Ltd., Taiwan) (which was used as an active pharmaceutical ingredient) was dissolved in the acidic first mixture at room temperature, followed by adjusting the pH value of the aqueous glucagon solution thus obtained to 2.7 with a 0.05 N HCl solution, so that an acidic aqueous glucagon solution is formed.

A suitable amount of WFI was mixed homogeneously with the acidic aqueous glucagon solution, followed by stirring at 200 rpm for 10 minutes, so as to obtain a second mixture (about 796 g to 804 g). The second mixture was subjected to sterile filtration using a polyethersulfone (PES) membrane capsule filter (pore size: 0.22 μm), and the resultant filtrate was collected, followed by freeze-drying, so as to obtain a water-dispersible freeze-dried powder containing glucagon.

The above preparation processes were conducted twice, thereby obtaining two water-dispersible freeze-dried powders (i.e., samples A and B).

### Example 2

### In Vitro Cell-Based Bioidentity Test of Water-Dispersible Freeze-Dried Powder Containing Glucagon

### Materials:

1. Medium A: Dulbecco's Modified Eagle Medium (DMEM) containing Glutamax, 10% (v/v) fetal bovine serum, and 0.5 mg/mL Geneticin® Selective Antibiotic (G418 Sulfate).
2. Medium B: Krebs' salt solution containing 0.3% (v/v) human serum albumin (10% HAS in water), 25 mM HEPES (4-(2-hydroxyethyl)-1-piperazineethanesulfonic acid), 1.7 mM 3-isobutyl-1-methyl-xanthine, 0.2 mg/mL of glucose, 650 KIU/mL of aprotinin, and 0.0003% (v/v) tween 80 (1% tween 80 in water), and having pH 7.4.
3. Medium C: Krebs' salt solution containing 0.3% (v/v) human serum albumin (10% HSA) and 25 mM HEPES, and having pH 7.4.
4. Buffer A: Hank's balanced salt solution containing 5.3 mM potassium chloride, 0.4 mM potassium phosphate monobasic, 4.2 mM sodium bicarbonate, 137.9 mM sodium chloride, 0.3 mM sodium phosphate dibasic anhydrous, and 5.6 mM dextrose.

### Procedures:

### A. Preparation of Standard Stock Solution

Recombinant glucagon (rGlucagon), United States Pharmacopeia (USP) reference standard (RS), was dissolved in water to a concentration of 4 mg/mL, followed by gently mixing through a rotator for 10 minutes or until being completely clear. The resultant solution was diluted 1000-fold with medium B to a concentration of 0.4 μg/mL, followed by 200-fold dilution with medium B, so as to obtain a standard stock solution containing 20 ng/mL rGlucagon.

### B. Preparation of Standard Solutions

The standard stock solution obtained in section A of this example was subjected to serial dilution with medium B, so as to obtain six standard solutions (i.e., standard solutions R8 to R3) having different concentrations (4 ng/mL, 0.8 ng/mL, 160 pg/mL, 32 pg/mL, 6.4 pg/mL, and 1.3 pg/mL). A final dilution process was made using the standard solution R3 with medium B to obtain a standard solution R2 having a concentration of 65 fg/mL. Furthermore, medium B was used as a blank solution R1.

### C. Preparation of Sample Solutions

A respective one of the samples A and B prepared in the above Example 1 was mixed with a suitable amount of water to reach a final glucagon concentration of 4 mg/mL. The resultant stock solutions A and B were subjected to serial dilution according to the procedure described in section B of this example, so as to obtain seven sample A solutions (i.e., sample solutions A2 to A8) and seven sample B solutions (i.e., sample solutions B2 to B8).

D. Preparation of cAMP Standard Solutions

A cAMP (cyclic adenosine monophosphate) solution (25 μM) was subjected to serial dilution with medium B, so as to obtain eight cAMP standard solutions (i.e., cAMP standard solutions C1 to C8) as shown in Table 1. Furthermore, medium B was also used as a blank solution C9.

TABLE 1

| | | Fold of | | | |
|---|---|---|---|---|---|
| Starting cAMP | | dilution | Final | | Resultant |
| concentration | | with medium B | concentration | | solution |
| 25 | μM | — | 25 | μM | C1 |
| 25 | μM | 3.3 | 7.5 | μM | C2 |
| 25 | μM | 10 | 2.5 | μM | C3 |
| 7.5 | μM | 10 | 0.75 | μM | C4 |
| 2.5 | μM | 10 | 250 | nM | C5 |
| 0.75 | μM | 10 | 75 | nM | C6 |
| 20 | nM | 10 | 25 | nM | C7 |
| 75 | nM | 10 | 7.5 | nM | C8 |

| | | | |
|---|---|---|---|
| Medium B only | — | No cAMP | C9 |

### E. Preparation of Donor Biotin-cAMP Beads

10 nmol of biotinylated cAMP was dissolved in 0.5 mL of phosphate buffered saline (pH 7.4), so as to obtain a biotinylated cAMP solution. 80 μL of the biotinylated cAMP solution was mixed with 33.8 mL of a 5 mM HEPES lysis buffer (containing 0.1% (w/v) BSA and 0.3% (v/v) polysorbate 20, and having a pH of 7.4), followed by adding 270 μL of donor beads and 2.4 mL of a 120 mM MgCl₂ solution, so as to obtain donor biotin-cAMP beads.

### F. Preparation of Cell Culture

A cell suspension of a glucagon receptor cell line (ATCC® CRL-3423™) was added to a sterile test tube containing 10 mL of warmed medium A, followed by homogeneously mixing. The resultant mixture was subjected to centrifugation at 125 g for 5 minutes. Thereafter, the supernatant was removed, and the cell pellet thus obtained was sufficiently suspended with fresh, warmed medium A. The resultant cell suspension was cultivated in a humidified incubator (37° C., 5% CO₂). Medium change was performed every two to three days. Cell passage was performed when the cultured cells reached 80%-90% of confluence. After a minimum of 8 passages, the glucagon receptor cells were used for the following experiment.

### G. Luminescence Analysis

The glucagon receptor cells obtained in section F of this example were suspended with fresh, warmed medium A, so as to obtain a cell suspension having a cell concentration of 4×10⁴ to 5×10⁴ cells/mL. Thereafter, 1 mL of the cell suspension was incubated in a respective well of a 96-well culture plate, followed by cultivation in an incubator (37° C., 5% CO₂) overnight. Medium A in each well was removed, followed by washing the cells with medium C. The cells were divided into 22 groups, including eight standard groups (i.e., standard groups R1 to R8) and fourteen experimental groups (i.e., experimental groups A2 to A8 and B2 to B8).

20 μL of anti-cAMP-acceptor beads was added to each group. Afterwards, 30 μL of a respective one of the standard solutions R1 to R8 obtained in section B of this example was added to a corresponding one of the standard groups R1 to R8, 30 μL of a respective one of the sample solutions A2 to A8 obtained in section C of this example was added to a corresponding one of the experimental groups A2 to A8, and 30 μL of a respective one of the sample solutions B2 to B8 obtained in section C of this example was added to a corresponding one of the experimental groups B2 to B8.

Furthermore, to each well of a 96-well culture plate containing no cells was added 20 μL of anti-cAMP-acceptor beads, followed by adding 30 μL of a respective one of the cAMP standard solutions C1 to C9 obtained in section D of this example, so as to obtain nine cAMP standard groups C1 to C9.

Each group of the cells was kept in the dark, and was incubated in a constant-temperature shaking incubator (37° C., 5% CO₂) for 35 to 60 minutes. 60 μL of the donor biotin-cAMP beads obtained in section E of this example was added to the cell culture of each group. Thereafter, the 96-well culture plates were wrapped with foil, followed by gently shaking of the 96-well culture plates at room temperature for 30 minutes. Each resultant mixture was subjected to determination of luminescence using a plate reader at an excitation wavelength of 680 nm and an emission wavelength of 520 to 620 nm.

The values thus obtained were used to determine USP rGlucagon content (units/mg) according to USP-NF <123> glucagon bioidentity tests. Briefly, the relative potency of glucagon was calculated using statistical methods for parallel-curve analysis with a 4-parameter logistic fit using all replicate values. The relative potency of the sample was determined by comparing the reference standard curve to the sample curve. The potency of the sample was calculated by multiplying the relative potency result by the potency of USP rGlucagon RS. The requirement of bioidentity is satisfied if the potency of the sample is not less than (NLT) 0.80 USP rGlucagon units/mg.

The experimental result shows that the sample A contained 1.14 USP rGlucagon units/mg, and the sample B contained 1.40 USP rGlucagon units/mg. This result indicates that the water-dispersible freeze-dried powder containing glucagon according to the present disclosure has great biologic activity, chemical stability, and physical stability, and hence can be used as a therapeutic agent for treating diabetes or hypoglycemia.

In the description above, for the purposes of explanation, numerous specific details have been set forth in order to provide a thorough understanding of the embodiments. It will be apparent, however, to one skilled in the art, that one or more other embodiments may be practiced without some of these specific details. It should also be appreciated that reference throughout this specification to “one embodiment,” “an embodiment,” an embodiment with an indication of an ordinal number and so forth means that a particular feature, structure, or characteristic may be included in the practice of the disclosure. It should be further appreciated that in the description, various features are sometimes grouped together in a single embodiment, figure, or description thereof for the purpose of streamlining the disclosure and aiding in the understanding of various inventive aspects, and that one or more features or specific details from one embodiment may be practiced together with one or more features or specific details from another embodiment, where appropriate, in the practice of the disclosure.

While the disclosure has been described in connection with what are considered the exemplary embodiments, it is understood that this disclosure is not limited to the disclosed embodiments but is intended to cover various arrangements included within the spirit and scope of the broadest interpretation so as to encompass all such modifications and equivalent arrangements.

What is claimed is:

1. A water-dispersible dry powder formulation comprising, based on the total weight of the water-dispersible dry powder formulation:
0.2 wt % to 4.0 wt % of glucagon;
10 wt % to 95.0 wt % of lactose;
0.001 wt % to 5.0 wt % of acetone; and
0.1 wt % to 10.0 wt % of water;
wherein the water-dispersible dry powder formulation has a pH value ranging from 2.0 to 6.0.

2. The water-dispersible dry powder formulation according to claim 1, wherein glucagon is present in an amount ranging from 0.5 wt % to 3.5 wt %.

3. The water-dispersible dry powder formulation according to claim 1, wherein lactose is present in an amount ranging from 20 wt % to 90 wt %.

4. The water-dispersible dry powder formulation according to claim 1, wherein acetone is present in an amount ranging from 0.5 wt % to 3.5 wt %.

5. The water-dispersible dry powder formulation according to claim 1, wherein water is present in an amount ranging from 0.5 wt % to 5.0 wt %.

6. The water-dispersible dry powder formulation according to claim 1, wherein the water-dispersible dry powder formulation has a pH value ranging from 2.0 to 4.0.

7. A method for producing a water-dispersible dry powder formulation, comprising:
a) admixing lactose with water at a temperature ranging from 30° C. to 40° C. to obtain an aqueous lactose solution;
b) admixing the aqueous lactose solution with acetone at room temperature, followed by adjusting a pH value of a first mixture thus obtained to pH 2.0 to 4.0 with an acidic aqueous solution, so that an acidic first mixture is formed;
c) admixing glucagon with water at room temperature, followed by adjusting a pH value of an aqueous glucagon solution thus obtained to pH 2.0 to 4.0 with another acidic aqueous solution, so that an acidic aqueous glucagon solution is formed; and
d) admixing the acidic first mixture with the acidic aqueous glucagon solution to obtain a second mixture, followed by subjecting the second mixture to a drying treatment to obtain the water-dispersible dry powder formulation.

8. The method according to claim 7, wherein each of the acidic aqueous solutions used in steps (b) and (c) is an aqueous solution containing hydrochloric acid, formic acid, or acetic acid.

9. The method according to claim 8, wherein the acidic aqueous solutions used in steps (b) and (c) are the same or different.

10. The method according to claim 7, wherein in step (d), the drying treatment is freeze-drying.

11. The method according to claim 7, wherein the water-dispersible dry powder formulation obtained from step (d) has a pH value ranging from 2.0 to 6.0 and comprises, based on the total weight of the water-dispersible dry powder formulation:
0.2 wt % to 4.0 wt % of glucagon;
10 wt % to 95.0 wt % of lactose;
0.001 wt % to 5.0 wt % of acetone; and
0.1 wt % to 10.0 wt % of water.
